(19) United States
(12) Patent Application Publication
(10) Pub. No.: US 2021/0077811 A1
(43) Pub. Date: Mar. 18, 2021

(54) WEARABLE LASER PAIN RELIEF DEVICE

(71) Applicant: LASINMED CORPORATION, New Taipei City (TW)

(72) Inventors: Sung-Ho Wong, Taoyuan City (TW); Tsung-Yu Chen, Tainan City (TW)

(21) Appl. No.: 16/568,326

(22) Filed: Sep. 12, 2019

(51) Int. Cl. A61N 1/36; A61B 5/00

(52) U.S. Cl. CPC A61N 1/36021; A61N 2005/0645; A61B 5/6802

(57) ABSTRACT

A wearable laser pain relief device contains: a power supply unit, a start unit, a sensing unit, and a drive unit. The power supply unit includes at least one battery and a charging device. The start unit is configured to be pressed by a user to turn on/off and to wake up the wearable laser pain relief device. The sensing unit includes an infrared transmitter and an infrared sensor, and the sensing unit is electrically connected with the start unit. The drive unit is electrically connected with the sensing unit and is configured to receive command signal(s) from the start unit so as to transmit the infrared ray to user's muscle, nerve, and tendon from the sensing unit by ways of a laser head of the drive unit.

### FIELD OF THE INVENTION

The present invention relates to a wearable laser pain relief device which is applied to soothe pain of user's muscle effectively, safety, and easily.

### BACKGROUND OF THE INVENTION

A conventional wearable laser pain relief device is fixed on user's skin to heat or to transmit currents to the skin or muscle tissue underneath it. By heating or using currents, the muscles are relaxed or contracted in such a way that the muscles produce a specific response, which helps the body to soothe or stimulate the muscles and muscle groups

The principle of the far-infrared wear-assisted device is mainly to mix the magnet with the far-infrared rays into the worn strap to provide the user with the wearer, but the current scientific data shows that the far-infrared rays have a soothing effect on the muscle muscles of the human body. It is not specific, and most of them need to increase the use of electronic heating devices. However, electronic heating devices require high-power current. Therefore, the far-infrared heating and heating-assisted soothing devices generally have to be connected to make the device bulky. Therefore, the design is only suitable for use in a sitting or lying position at home, and it is not convenient to wear out.

Another conventional wearable laser pain relief device is used to stimulate the nerve reaction of human muscles through the release of current, in principle, it is forced muscle contraction movement rather than soothing. This type of product has the effect of auxiliary training. Although the device design can be designed to be small and then wear out, it is not for use in sore areas, nor soothing.

The present invention has arisen to mitigate and/or obviate the afore-described disadvantages.

### SUMMARY OF THE INVENTION

The primary aspect of the present invention is to provide a wearable laser pain relief device which is applied to soothe pain of user's muscle effectively, safety, and easily.

Another aspect of the present invention is to provide a wearable laser pain relief device which is durable.

To obtain the above aspect, a wearable laser pain relief device provided by the present invention contains: a power supply unit, a start unit, a sensing unit, and a drive unit.

The power supply unit includes at least one battery and a charging device, and the power supply unit is electrically connected with and supplies power to the start unit, the sensing unit, and the drive unit.

The start unit is configured to be pressed by a user to turn on/off and to wake up the wearable laser pain relief device.

The sensing unit includes an infrared transmitter and an infrared sensor, and the sensing unit is electrically connected with the start unit. When the start unit is switched to a start mode and a wake up mode, the infrared transmitter of the sensing unit sends infrared ray to irradiate user's skin, and the infrared sensor detects reflection of the infrared ray from the user's skin so as to judge a length of the infrared ray between the infrared transmitter and the user's skin, and when the length of the infrared ray is more than a predetermined length, the sensing unit controls the start unit to turn off.

The drive unit is electrically connected with the sensing unit and is configured to receive command signal(s) from the start unit so as to transmit the infrared ray to user's muscle, nerve, and tendon from the sensing unit by ways of a laser head of the drive unit.

### BRIEF DESCRIPTION OF THE DRAWINGS

FIG. 1 is a flow chart view showing the operation of a wearable laser pain relief device according to a preferred embodiment of the present invention.

FIG. 2 is a perspective view showing the assembly of the wearable laser pain relief device according to the preferred embodiment of the present invention.

FIG. 3 is another perspective view showing the assembly of the wearable laser pain relief device according to the preferred embodiment of the present invention.

FIG. 4 is a perspective view showing the operation of the wearable laser pain relief device according to the preferred embodiment of the present invention.

FIG. 5 is a side plan view showing the application of the wearable laser pain relief device according to the preferred embodiment of the present invention.

### DETAILED DESCRIPTION OF THE PREFERRED EMBODIMENTS

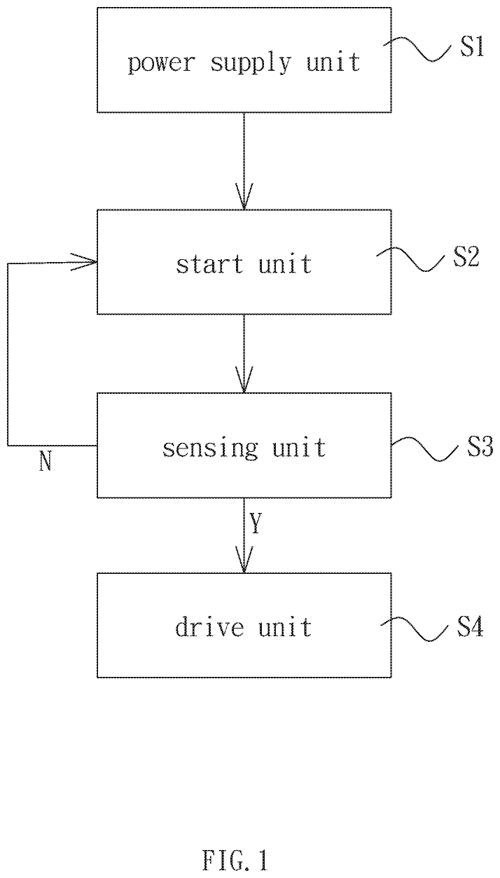

With reference to FIG. 1, a wearable laser pain relief device according to a preferred embodiment of the present invention is applied to soothe pain of user's muscle effectively, safety, and easily.

The wearable laser pain relief device 10 comprises: a power supply unit S1, a start unit S2, a sensing unit S3, and a drive unit S4.

The power supply unit S1 includes at least one battery and a charging device, wherein the power supply unit S1 is electrically connected with and supplies power to the start unit S2, the sensing unit S3, and the drive unit S4.

The start unit S2 is configured to be pressed by a user to turn on/off and to wake up the wearable laser pain relief device, and the start unit S2 includes a vibration element for producing a vibration.

The sensing unit S3 includes an infrared transmitter and an infrared sensor, wherein the sensing unit S3 is electrically connected with the start unit S2. When the start unit S2 is switched to a start mode and a wake up mode, the infrared transmitter of the sensing unit S3 sends infrared ray to irradiate user's skin, and the infrared sensor detects reflection of the infrared ray from the user's skin so as to judge a length of the infrared ray between the infrared transmitter and the user's skin, and when the length of the infrared ray is more than a predetermined length, the sensing unit S3 controls the start unit S2 to turn off. In this embodiment, when the length of the infrared ray is more than 1 cm, the sensing unit S3 controls the start unit S2 to turn off so that laser lights do not irradiate user's eyes.

The drive unit S4 is electrically connected with the sensing unit S3 and is configured to receive command signal(s) from the start unit S2 so as to transmit the infrared ray to user's muscle, nerve, and tendon from the sensing unit S3 by ways of a laser head of the drive unit S4, thus soothing pain of user's muscle and facilitating blood circulation.

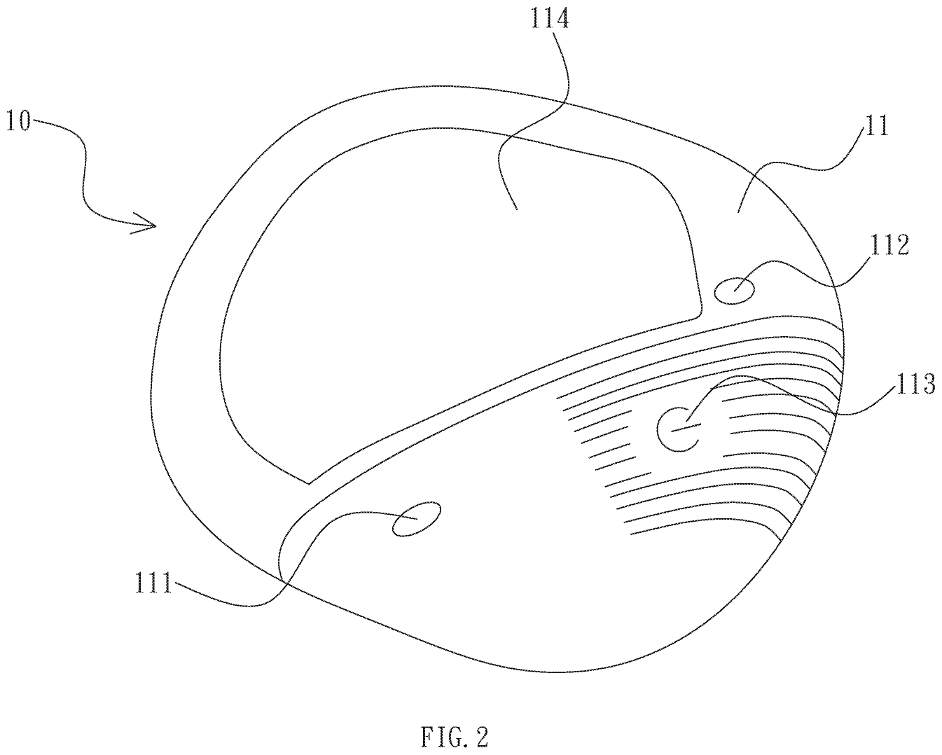

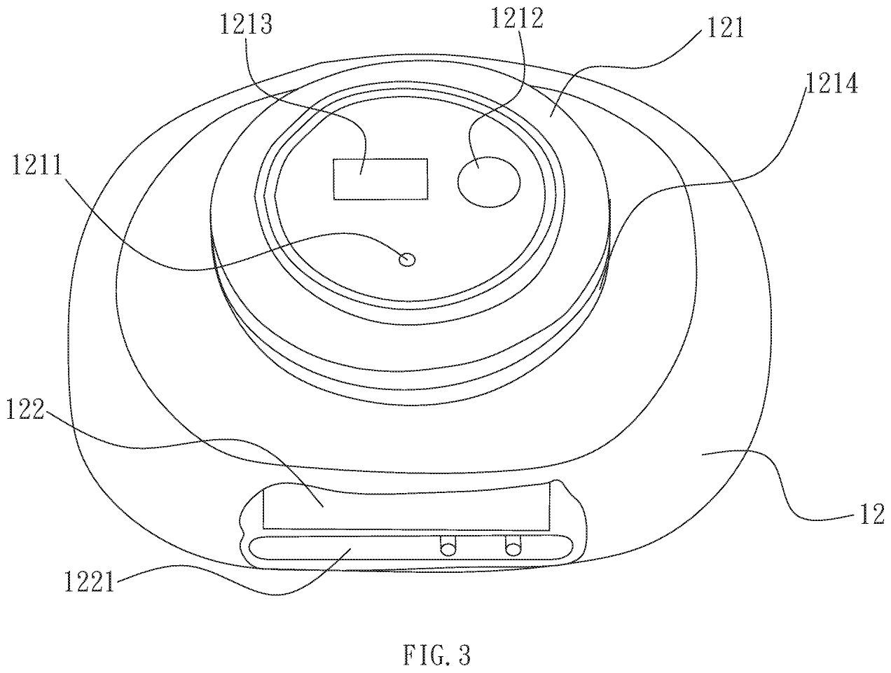

Referring to FIGS. 2 and 3, the wearable laser pain relief device 10 further comprises: a first body 11 and a second body 12 connected with the first body 11, wherein the first body 11 and the second body 12 accommodate a circuit board corresponding to the power supply unit S1, the start unit S2, the sensing unit S3, and the drive unit S4. The first body 11 and the second body 12 include a waterproof structure so as to avoid user's sweat wetting the circuit board.

The first body 11 includes a start switch 111, an indicator light 112, a wake up switch 113, and the vibration element 114, wherein the start switch 111 controls the start unit S2 to turn on/off, the indicator light 112 produces light signals so that the user judges whether the start switch 111 turns on or off, the wake up switch 113 is configured to control the start unit S2 to stop the laser lights. Preferably, the indicator light 112 produces the light signals so that the user judges whether the wake up switch 113 turns on or off. The first body 11 includes the vibration element 114 selectively mating with the indicator light 112 to vibrate so that the user knows operation status when he/she does not view the light signals.

The second body 12 includes a contact portion 121 arranged on a bottom thereof, and the second body 2 includes a power charge portion 112 fixed on a side thereof, wherein the contact portion 121 extends out of the second body 12 so as to be fixed on a desired position of the user′ skin by the user, thus irradiating the laser lights to the desired position accurately. The contact portion 121 has the laser head 1211, the infrared transmitter 1212, and the infrared sensor 1213, wherein the infrared transmitter 1212 and the infrared sensor 1213 are connected with the sensing unit S3 so that the infrared transmitter 1212 transmits the infrared ray to the user's skin, and the infrared sensor 1213 detects the length of the infrared ray between the infrared transmitter 1212 and the user's skin. The laser head 1211 is connected with the drive unit S4 and is controlled to transmit the laser lights to the user's skin within the predetermined length, and the contact portion 121 has a locking groove 1214 defined around a peripheral side of the second body 12.

The second body 12 includes the power charge portion 122 having a waterproof charger 1221, wherein the power charge portion 122 is electrically connected with the at least one battery and the charging device of the power supply unit S1 so as to charge the power.

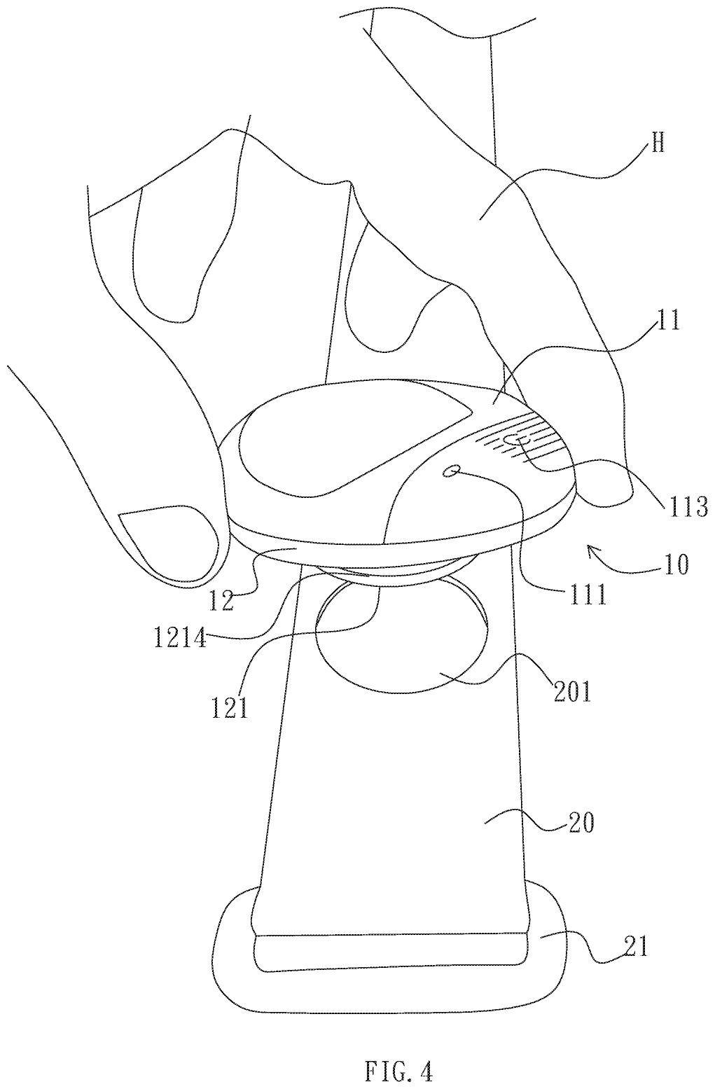

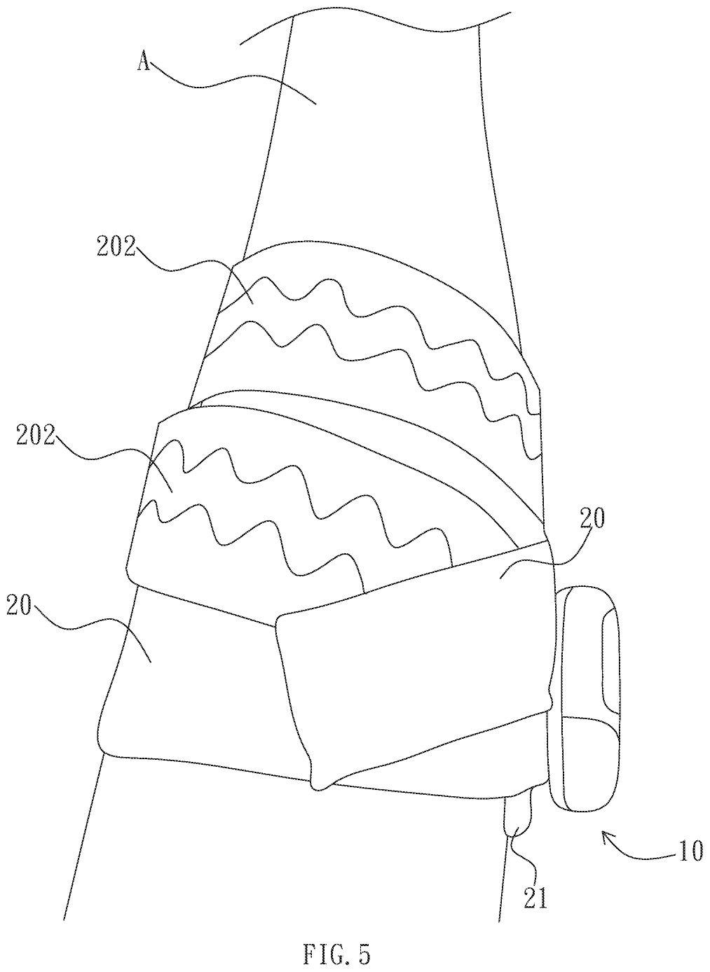

As shown in FIGS. 4 and 5, the wearable laser pain relief device 10 comprises a fixing element (such as a band) 20 on which a hook and loop fastener 202 is mounted so as to fix on the user. The band 20 includes a fastener 21 arranged on an end thereof and configured to fasten with the hook and loop fastener 202, after winding the user and being inserted into the fastener 21. Preferably, the fixing element 20 is a silicone tape for adhering on the user. The fixing element 20 has an orifice 201 for fitting with the locking groove 1214 so that the contact portion 121 is aligned with and is mounted on the fixing element 20 to irradiate the laser lights to user's elbow A after extending out of the second body 12, and the orifice 201 is fitted with the locking groove 1214 and faces outward relative to the first body 11. The start switch 111 and the wake up switch 113 are operated by user's hand H, and the locking groove 1214 is removed from the orifice 201 so as to change different sizes of the fixing element 20 (i.e., the bands) based on using requirements.

While the preferred embodiments of the invention have been set forth for the purpose of disclosure, modifications of the disclosed embodiments of the invention as well as other embodiments thereof may occur to those skilled in the art. The scope of the claims should not be limited by the preferred embodiments set forth in the examples, but should be given the broadest interpretation consistent with the description as a whole.

What is claimed is:

1. A wearable laser pain relief device (10) comprising: a power supply unit (S1), a start unit (S2), a sensing unit (S3), and a drive unit (S4);
wherein the power supply unit (S1) includes at least one battery and a charging device, and the power supply unit (S1) is electrically connected with and supplies power to the start unit (S2), the sensing unit (S3), and the drive unit (S4);
wherein the start unit (S2) is configured to be pressed by a user to turn on/off and to wake up the wearable laser pain relief device (10);
wherein the sensing unit (S3) includes an infrared transmitter (1212) and an infrared sensor (1213), and the sensing unit (S3) is electrically connected with the start unit (S2); when the start unit (S2) is switched to a start mode and a wake up mode, the infrared transmitter (1212) of the sensing unit (S3) sends infrared ray to irradiate user's skin, and the infrared sensor (1213) detects reflection of the infrared ray from the user's skin so as to judge a length of the infrared ray between the infrared transmitter (1212) and the user's skin, and when the length of the infrared ray is more than a predetermined length, the sensing unit (S3) controls the start unit (S2) to turn off;
wherein the drive unit (S4) is electrically connected with the sensing unit (S3) and is configured to receive command signal(s) from the start unit (S2) so as to transmit the infrared ray to user's muscle, nerve, and tendon from the sensing unit (S3) by ways of a laser head (1211) of the drive unit (S4).

2. The wearable laser pain relief device (10) as claimed in claim 1, wherein when the infrared sensor (1213) detects the length of the infrared ray is more than 1 cm, the sensing unit (S3) controls the start unit (S2) to turn off so that laser lights do not irradiate user's eyes.

3. The wearable laser pain relief device (10) as claimed in claim 1, wherein the start unit (S2) is electrically connected with the power supply unit (S1) so as to switch to the wake up mode.

4. The wearable laser pain relief device (10) as claimed in claim 1, wherein the start unit (S2) includes a vibration element for producing a vibration.

5. A wearable laser pain relief device (10) comprising:
a first body (11) including a start switch (111), an indicator light (112), wherein the start switch (111) controls a start unit (S2) to turn on/off, the indicator light (112) produces light signals so that a user judges whether the start switch (111) turns on or off;
a second body (12) including a contact portion (121) arranged on a bottom of the second body (12) and irradiating laser lights to a desired position of user's skin, the contact portion (121) having a laser head (1211), an infrared transmitter (1212), and an infrared sensor (1213), wherein the infrared transmitter (1212) and the infrared sensor (1213) are connected with the sensing unit (S3) so that the infrared transmitter (1212) transmits infrared ray to the user's skin, and the infrared sensor (1213) detects a length of the infrared ray between the infrared transmitter (1212) and the user's skin, the laser head (1211) is connected with the drive unit (S4) and is controlled to transmit the laser lights to the user's skin within a predetermined length;
a fixing element (20) configured to fix the contact portion (121) of the wearable laser pain relief device (10) on the user.

6. The wearable laser pain relief device (10) as claimed in claim 5, wherein the first body (11) includes a wake up switch (113) configured to control the start unit (S2) to stop the laser lights, and the indicator light (112) produces light signals so that the user judges whether the wake up switch (113) turns on or off.

7. The wearable laser pain relief device (10) as claimed in claim 5, wherein the first body (11) includes a vibration element (114) selectively mating with the indicator light (112) to vibrate so that the user knows operation status when the user does not view the light signals.

8. The wearable laser pain relief device (10) as claimed in claim 5, wherein the contact portion (121) extends out of the second body (12) so as to be fixed on the desired position of the user′ skin by the user, thus irradiating the laser lights to the desired position accurately.

9. The wearable laser pain relief device (10) as claimed in claim 5, wherein the second body (12) includes a power charge portion (122) having a waterproof charger (1221), and the power charge portion (122) is electrically connected with at least one battery and a charging device of the power supply unit (S1) so as to charge a power.

10. The wearable laser pain relief device (10) as claimed in claim 5, wherein the contact portion (121) has a locking groove (1214) defined around a peripheral side of the second body (12), and the fixing element (20) has an orifice (201) for fitting with the locking groove (1214).
